(19) United States
(12) Patent Application Publication
(10) Pub. No.: US 2004/0102409 A1
(43) Pub. Date: May 27, 2004

(54) Medicament for locally treating or preventing a chemotherapy-induced mucositis and other side effects resulting from chemotherapy

(76) Inventors: Werner Geurtsen, Hannover (DE); Ray Monnat, Seattle, WA (US); Laurence Loeb, Bellevue, WA (US)

Correspondence Address: Robert E Muir, Husch & Eppenberger, Suite 1400, 401 Main Street, Peoria, IL, 61602-1241, US

(21) Appl. No.: 10/450,629

(22) PCT Filed: Dec. 17, 2001
(86) PCT No.: PCT/DE01/04864

(30) Foreign Application Priority Data
Dec. 15, 2000 (DE) 100 62 766.8

(51) Int. Cl. A61K 48/00; A61K 38/48

(52) U.S. Cl. 514/44; 424/94.63

(57) ABSTRACT

The invention relates to a medicament for locally treating or preventing a mucositis, which is induced by chemotherapy with thymidylate synthase inhibitors, in the oral cavity and in the gastrointestinal tract, whereby a thymidylate synthase mutant, which is coupled to a transductor for the transduction into a cell, which has an intact enzyme activity, and which is resistant to thymidylate synthase inhibitors, is administered.

The invention concerns a drug for the local treatment or prevention of a mucositis, which is induced by a chemotherapy with inhibitors of thymidylate synthase (in the following abbreviated as TS), specifically in the gastrointestinal tract including the oral cavity, and other adverse effects due to TS inhibitors, which can be treated locally. Generally, the invention concerns the application of specific TS mutants for this purpose.

TS-inhibitors are used for the chemotherapy of various types of cancers for some time, in order to prevent the new generation of cancerous tissues by inhibiting the DNA synthesis in these cells. TS catalyses the speed determining step during the de novo biosynthesis of thymidylate, an important component of DNA-synthesis and -repair. Common TS inhibitors are for example 5-fluorouracil (5-FU), fluorodeoxyunridylate (FdUMP) and various folate analoga (FA), which replace the TS substrates and thus block TS, which then inhibits the DNA synthesis.

Unfortunately, TS inhibitors cannot be administered well-aimed in a sufficient manner, so that healthy cells are also damaged, which then causes severe side effects.

A mucositis of the gastrointestinal tract including the oral cavity is frequently associated with the chemotherapy using TS inhibitors. The effects of a mucositis can be so severe that the chemotherapy has to be stopped or shortened or the optimum dosage of the chemotherapy has to be reduced, which impairs the prognosis of the cancer therapy. Additionally, more infections are associated with a damaged mucosa, which cause an increased morbidity, dietary problems and a drastically reduced quality of life of the patients. Presently, such a mucositis can be only treated symptomatically with little success.

The thymidylate synthase is known for a long period of time and was described for instance by Carreras et al [1]. Human TS mutants have been also reported, which are resistant to Thymitaq™ (AG337) and 5-fluoro-2-deoxyuridylate. Such mutants were investigated for instance within the scope of mechanistic studies about the DNA synthesis [2].

A human thymidylate synthase, which is mutated at amino acid residues 49, 52, 108, 221, and 225 was disclosed in WO 98/33518 [3]. Aim of the authors is to understand the blocking and resistance mechanisms using specific mutations of the thymidylate synthase based on the knowledge of its three dimensional structure, in order to develop new and better cancer drugs for the chemotherapy with TS inhibitors. For this, mutants using random and site directed mutation were produced, isolated, and characterized. It is also mentioned that the mutants may be potentially usable for gene therapy, in order to protect specific normal cells or to serve as selection marker in therapeutic gene transfer protocols. However, no usable gene therapeutic procedure is being given.

Compared with this the object of the present invention is to provide a drug for the treatment or prevention of a chemotherapy-induced mucositis and other side effects associated with a chemotherapy available, which has a causal effect instead of being symptomatically effective. The drug shall be specifically locally applicable in order to be able to administer it directly to the affected tissues.

In order to solve this problem it is intended, to design a drug for the local treatment or prevention of a mucositis or other side effects in the gastrointestinal tract including the oral cavity, which are caused by chemotherapy with thymidylate synthase inhibitors. This drug will contain at least one thymidylate synthase mutant either in form of a protein or a nucleic acid. The thymidylate synthase mutant will be linked to a transducer, which causes the intake of the drug into the cells that need to be treated. The thymidylate synthase mutant reveals a completely intact enzymatic activity and is resistant to thymidylate synthase inhibitors.

Contrary to the local application of merely symptomatically effective drugs, which are frequently ineffective for the treatment of a chemotherapy-associated mucositis, according to the present invention, active TS-mutants are used which are resistant to TS inhibitors and thus are causally effective against the cell destruction, which is causative for a mucositis.

An important aspect of this invention is, to infiltrate the TS mutants as easy, effective, and selective as possible into the target cells. Due to obvious reasons it must be avoided, to infiltrate TS mutants into tumor cells too, since this would interfere with the essential therapy, the chemotherapy with TS inhibitors. Therefore it is intended, to couple the TS mutants with a “transporter molecule”, the so-called transducer, which can mediate the transport, i.e. the transduction, into the target cell. The transduction shall be preferentially locally, i.e. specifically into the mucosa, which is affected by the chemotherapy, but under no circumstances systematically. For this, various possibilities are available.

Generally, the transduction of the protein or the gene can be performed with any appropriate method, which is known to the expert. Many transduction techniques have been described in the literature. This includes the use of cationic liposomes, viral vectors or the coupling with various transduction proteins or peptides in general.

A further possibility for the protein transduction is, to couple the TS mutants with a transporter protein, which can penetrate the biological cell membrane. During the past decade various transduction proteins (PTDs, Protein Transduction Domains) were found. This includes among others the TAT protein of HIV, the drosophila homeotic transcription factor (ANTP) and the herpes simplex virus type I (HSV-1) VP 22 transcription factor. Appropriate PTDs and their in vivo application have been already described [5]. It was possible, to deliver active enzymes and DNA into all tissues by means of PTDs, even passing the blood-brain-barrier. It could be demonstrated with more than 60 TAT-fusion proteins that a transport (a transduction) was successful in nearly 100% of the investigated cell types (primary and immortalized cells) as well in cells/tissues of mice within 5 minutes.

The transport of TAT fusion proteins in mammalian cells is described in laboratory manuals [6], TAT-derived transport polypeptides have been already disclosed [7]. These can deliver polypeptides and nucleic acids in the cytoplasm and the nucleus of cells in vitro and in vivo. The intracellular delivery of freight molecules is achieved by means of transport polypeptides, which comprise one or several parts of the HIV-TAT-protein and are covalently bound to the freight molecules.

According to the present invention, the drug is designed for the local application in the gastrointestinal tract including the oral cavity. A preferred type of preparation is an oral rinsing solution. Another preferred type of preparation are protease protected and acid resistant capsules, coated tablets (dragees), or granulates that contain the TS mutants and which are produced in a way that is familiar to the expert. These forms of administration allow the passage of the stomach without destruction of the transducer coupled TS mutants so that they can arrive intact in the intestine. This way the mucosa or related tissues of the intestine can be also protected by transduction of TS mutants against unwanted side effects of chemotherapeutic drugs, which contain TS inhibitors.

Another preferred type of administration of TS mutants for the therapy of the mucosa or related tissues of the intestine are rectally applied types of administration, such as clysmas (enemas) or suppositories, which allow the bypassing of the acidic gastric environment. The treatment of the gastric mucosa requires the pretreatment with oral acid blockers, which are used in medicine since many years for a short-term neutralization of the acidic environment. Acid sensitive drugs such as the transducer coupled TS mutants can be also administered to the mucosa of the stomach by means of an orally applied therapeutic subsequently to such a pretreatment.

Another preferred type of administration would be the application of viable bacteria in the intestinal micro flora of the patient under chemotherapy. These bacteria should be modified in such a way that the express the appropriate TAT-TS fusion proteins. The microbial strain, which is used for such a therapeutic strategy, should be selected in such a way that it can survive and proliferate in the physiological intestinal flora for at least several days. But on the other hand, a certain share of these bacteria should be also lyzed in the intestinal environment in order to liberate the TAT-TS-fusion proteins which are expressed by this microbial strain. Alternatively, other microorganisms, such as yeast, could be used, which are capable of secreting expressed proteins. In principle, all types of organisms are appropriate, which can survive in the human intestinal flora and which are compatible with human health. The bacteria could be applied either orally by means of acid-resistant capsules or rectally by means of suppositories.

Dependent of the type of administration various adjuvants, additives and fillers for the production of drugs, which contain transducer-coupled TS mutants can be applied. Oral rinsing solutions and clysmas (enemas) can be formulated as physiological saline solution. The pH of this solution can be adjusted to physiologic values for instance by means of potassium hydrogen phosphate and potassium-di-hydrogen phosphate. In order to secure the stability of the TS-mutants, accessory substances such as reducing agents or mild detergents, which are compatible with proteins, can be used.

Proteins, such as casein or human albumin, can be added to the drug for additional stabilization of the TS mutants. If necessary, preparations containing the transducer coupled TS mutants, which will be used as liquids, can be made available as dried powders and are dissolved in an appropriate diluent, such as water, immediately before application.

Other substances known to the skilled person, such as methacrylic acid polymer, macrogol, carnauba wax and other waxes, shellac, polyvidone, cellulose, talcum, calcium stearate, xanthane rubber, hard fats, silicon oxide and other appropriate adjuvants, additives or filler can be used. For instance, sorbic acid or sodium benzoate can be added as preservatives. Additional adjuvants or additives, which are known to the skilled person, can be added such as coloring, flavorring substances or fragrances.

The cells of the gastrointestinal tract (mucosa cells), which are affected by side effects due to a chemotherapy with TS inhibitors, can be locally reached particularly fast, efficient and selective. According to a presently preferred embodiment invention, protein mutants with a TAT transporter protein are infiltrated into the cells. According to the invention, this system is particularly appropriate, since animal experiments revealed that TAT fusion proteins can be transduced into primary cells within 5 minutes. Thus, TAT fusion proteins are specifically suitable for a topical application, for instance as oral rinsing solution. Transducers, which need a long period of time to fulfill their function, are generally less appropriate for a use in rinsing solutions. In another preferred embodiment Herpes VP22 fusion proteins can be used for the transduction of TS mutants instead of TAT fusion proteins. Further, transport proteins, which are optimized for transduction by means of site directed or random mutagenesis, can be applied.

Further, the non-covalent complex formation according to the Chariot™ protein transfection technique can be used in the sense of this invention [4]. The complexing substance allows the penetration of the biological cell membrane in a short period of time. Simultaneously, the transported protein is protected in the non-covalent complex. Due to the short transduction times up to 2 hours complex substances are specifically useful as transducers for the direct, local application of relatively short-lived protein mutants (life span minutes up to 24 hours). Other complexing substances, which are useful for the aforementioned purpose, are also applicable.

The invention includes the direct use of TS mutants as isolated proteins on the one hand, and their application as nucleic acids, which encode these TS mutants and thus make the expression of the TS mutants in the target cells possible on the other hand. Potential nucleic acids for example would be mRNA of TS mutants or expression vectors for TS mutants with constitutively active and inducible promoters. Since there is only a transient expression of the TS mutants necessary for the treatment of chemotherapy-associated side effects, only those systems should be used in case of expressions vectors, which are only active for a few days or weeks and which are not incorporated into the genome of the patient. In order to avoid this problem TS mutants could be preferentially transduced as proteins or mRNA. The infiltrated or in the cells generated TS mutants display their physiological functions in the (mucosa) tissue preventively or as therapeutic of already damaged cells. Their special feature is that they are not or only little inhibited by the TS inhibitors, which are used for the chemotherapy. TS mutants as proteins as well as TS mutant coding nucleic acids can be coupled to the same transducers (complexing substance/PTDs). TS nucleic acids, which encode TS inhibitor resistant proteins, are preferentially transduced into the different types of cells of the mucosa or other target tissues by means of transport proteins (PTDs), which can be optimized for the specific genes concerning the transduction efficiency on the other hand.

The TS nucleic acid mutants will be produced by random or site directed mutagenesis and checked for the desired characteristics. The accompanying protein should reveal a good stability combined with a high enzymatic activity and resistance to TS inhibitors. The generation and characterization of TS mutants is comprehensively described for example in WO 98/33518, which is referred to regarding the experimental data of mutants.

The resistance of the single mutants can be tested with different TS inhibitors. Presently, 5-FU (5-fluorouracil), which is metabolized to FdUMP (fluorodeoxyuridylate), FdUMP or folate analoga, such as Raltitrexed™, Thymitaq™ or AG331 are mainly applied. Therefore, the mutants will be tested for resistance to these substances. Additional Ts inhibitors can be included in necessary.

The TS mutants are preferentially generated by means of cloning cDNA of resistant TS mutants in bacterial expression vectors [6].

As further stage of this invention it is also intended that the transduction efficiency of the transport proteins, which are used in this invention, will be optimized. The transductions efficiency arises from the speed of penetration of the fusion protein, which consists of the particular PTD and the TS mutant and the portion of molecules which successfully infiltrated the cells. This can be achieved by random or site directed mutagenesis of the particular PTD and subsequent analysis of the transduction characteristics.

The success of the proposed application will be significantly improved by optimizing the transport protein, i.e. the transducer, towards the best possible transduction efficiency for the particular TS mutant. It is obvious that the success of a treatment of a mucositis is essentially dependent on infiltrating the target cells with the highest possible amount of nucleic acid or protein mutants as fast as possible and maintaining the function or activity of the mutant. It is essential for this goal to optimize the PTD.

It is referred to the information and data of all papers, which are cited in this application.

The invention will be demonstrated using the following examples with reference to Figures:

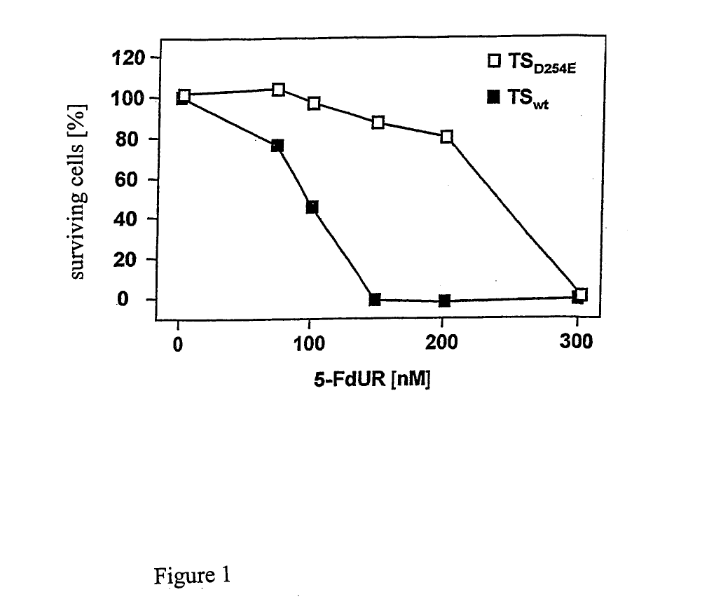

FIG. 1: Graph showing the 5-FdUR (5-fluorodeoxyuridine=active metabolite, which is generated of 5-fluorouracil in the cells) resistance of wild type TS (TS_{WT}) compared to a TS mutant, where in position 254 the amino acid residue D (aspartic acid) was replaced by E (glutamic acid) (TS_{D254E}). The y-axis shows the survival rate of the bacteria in %, which were transfected with a TS-expression vector, referred to TS_{WT}-transfected bacteria in the absence of the TS-inhibitor 5-FdUR. The x-axis shows the applied concentrations of 5-FdUR. 80% of the TS mutants TS_{D254E} survive even at 200 nM 5-FdUR whereas no wild type TS_{WT} survived at this 5-FdUR concentration.

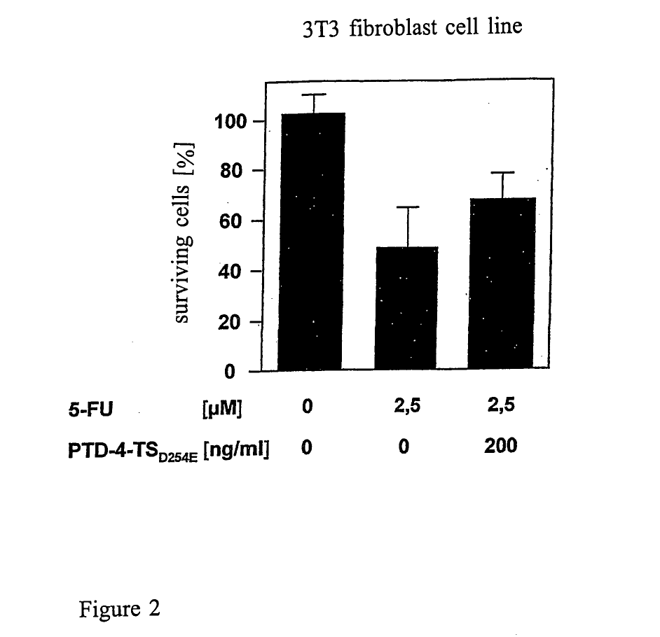

FIG. 2: Graph showing the survival rate in % of 3T3 fibroblasts incubated with 2.5 μM 5-FU (5-fluorouracil) after 24 h dependent on the presence or absence of 200 ng/ml of the TS mutant TS_{D254E}. Since 2.5 μM 5-FU represent the ED₅₀ concentration of this drug for 3T3 cells, only 50% of the cells survived in the absence of PTD-4-TS_{D254E} (2. column from left). However, 70% of the cells, which were also treated with PTD-4-TS_{D254E}, survived the chemotherapy with 5-FU (3. column from left). The left column shows the control, 100% of the 3T3 cells, which were not treated with 5-FU survived.

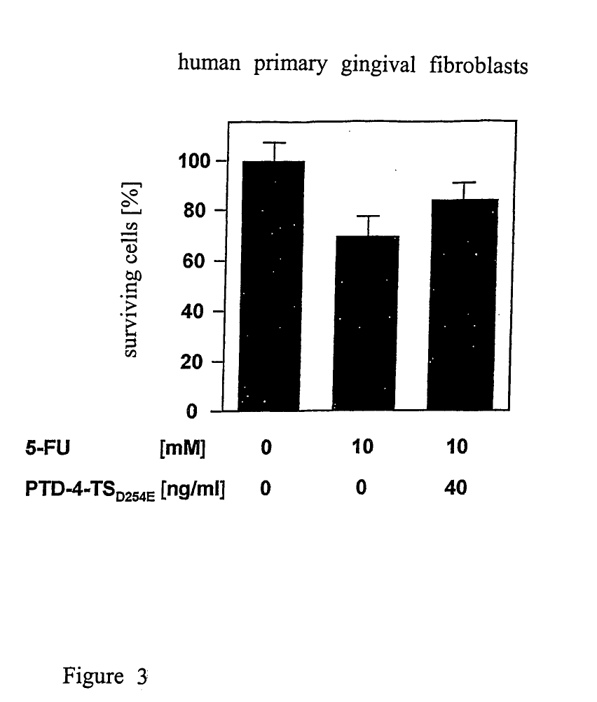

FIG. 3: Graph showing the survival rate of primary human gingival fibroblasts in % in the presence of 10 mM 5-FU after 24 h, either in presence or absence of 40 ng/ml of TS mutant TS_{D254E}. Primary gingival fibroblasts reveal a significantly higher ED₅₀ for 5-FU compared to the faster growing 3T3 cells due to their slower proliferation. 85% of the gingival fibroblasts, which were treated with PTD-4-TS_{D254E} survived the chemotherapy with 5-FU (3. column from left), whereas only 70% of the untreated gingival fibroblasts survived. The left column shows the control; 100% of gingival fibroblasts, which were not treated with 5-FU, survived.

### EXAMPLES

### Example 1

### Expression and Isolation of PTD-4-TS_{D254E}-Fusion Protein

The vector pTAT-HA, which has been already described (8) was used for the expression of the PTD-4-TS_{D254E}-fusion protein. The original TAT sequence (YGRKKRRQRRR) was exchanged in this vector by a TAT sequence (YARAAARQARA), which was optimized by random mutagenesis. The optimized TAT sequence has a 33fold more efficient transduction activity in comparison to the original TAT sequence and will be designated as PTD-4 (“protein transduction domain 4”) in the following (9). Into the vector pTAT-HA, which was modified in this way, the sequence of wild type thymidylate synthase TS_{WT} and of the TS mutant TS_{D254E}, respectively, were inserted under maintenance of their reading frame. The vector causes the expression of fusion proteins with 6 histidine residues N-terminal to the TAT-sequence. TAT-TS-fusion proteins were generated by transfection of BL21(DE3)pLysS E. coli cells (Novagen, Madison, Wis., USA) with this vector. For this purpose bacteria were cultured over night and the fresh culture was inoculated and incubated for 4 to 6 hours at 37° C. in a shaker incubator. Cells were separated from the medium by centrifugation and the cell pellet was lyzed with 10 mL buffer A (8 M urea, 20 mM HEPES, pH 8.0 and 100 mM sodium chloride). Unsoluble cell debris was separated by centrifugation und the clear supernatant was applied on to a Ni-NTA nickel chelate chromatography column (Qiagen, Valecia, Calif., USA). The column was washed with buffer A supplemented with 10 mM imidazol and the bound protein was eluted from the column in three steps with buffer A, which was supplemented with 100, 250, and 500 mM imidazol, respectively. The fractions, which contained the TAT-TS-fusion protein, were diluted 1:1 with a buffer, which contained 20 mM HEPES resulting in the following buffer composition: 4 M urea, 20 mM HEPES, pH 8.0, and 50 mM sodium chloride. The sample was applied on to a Mono Q 10/10 chromatography column (Pharmacia, Piscataway, N.J., USA) using a FPLC-chromatography device. Subsequently the column was rinsed with the same buffer, which did not contain urea, i.e., with 20 mM HEPES, 50 mM sodium chloride, pH 8.0 and the bound TAT-TS-fusion protein was eluted from the column with a buffer, which contained 1 M sodium chloride, 20 mM HEPES, pH 8.0. These purified fusion proteins were adjusted to a PBS-buffer by means of gel filtration with a PD10 sephadex G-25 gel filtration column (Pharmacia, Piscataway, N.J., USA), which determines the protein concentration with a Bradford protein assay in a way known to the skilled person. Then the protein was shock frozen with liquid nitrogen and stored at −80° C.

### Example 2

### Testing the 5-FU-resistance of TS-mutants

The bacterial strain χ2913 was transfected with expressions vectors of the TS-mutants and defined amounts of bacteria in the exponential growth phase were plated on selection plates. The selection plates contained increasing concentrations of 5-fluorodeoxyuridine (0, 75, 150, 200, respectively 300 nM). 5-fluorodeoxyuridine (5-FdUR) is a very effective metabolite, which is generated from 5-fluorouracil in the cell. The selection plates were made of minimal medium, which contained carbenicilline and tetracycline. Since the selection plates did not contain thymidine, only those bacteria can grow, which have a functioning thymidylate synthesis that is not inhibited by the concentration of the TS-inhibitor 5-FdUR present in the plates. After 36 to 48 hours the number of growing colonies was determined. For this the number of colonies of bacteria, which were transfected with wild type thymidylate synthase was set as 100% and all other numbers of colonies were correspondingly converted to % values.

### Example 3

### Treatment of 3T3-Fibroblast Cell Line with 5-FU and TAT-TS-Fusions Proteins

3T3 fibroblasts cells (available by the American Type Cell Collection (ATCC, Rockville, Md., USA) were seeded with a cell density of 1×10⁴ cells per well into 96-well-cell culture plates. DMEM medium with 10% fetal calve serum is used as cell culture medium, and cells are cultured in a CO₂-incubator at 37° C. and 5% CO₂. The cell culture medium is aspirated after 24 hours and substituted by 200 μL/well of the test assay and cells are incubated for another 24 hours. Subsequently the portion of viable cells is determined correspondingly to example 4. The test assays are composed as follows:

Controls without 5-FU and without PTD-4-TS_{D254E}-fusion protein: 200 μL/well of DMEM with 10%.fetal calve serum

Samples with 5-FU without PTD-4-TS_{D254E}-fusion protein: 200 μL/well of DMEM with 10% fetal calve serum and 2.5 μM 5-FU

Samples with 5-FU and PTD-4-TS_{D254E}-fusion protein (wild type TS or TS-mutants):

200 μL/well of DMEM with 10% fetal calve serum, 2.5 μM 5-FU and 40 ng/mL PTD-4-TS_{D254E}-fusion protein

The fusion protein PTD-4-TS_{D254E} was made similar to example 1.

### Example 4

### Determination of the Cytotoxicity of 5-FU in Fibroblasts in the Presence or Absence of TAT-TS-Fusion Proteins with the Dye Hoechst 33342

After completion of the 24-hour treatment of the cells in the control- or test assays similar to example 3 the medium is removed from the 96-well-plates and substituted by 200 μL/well DMEM medium with 10% fetal calve serum and 10 μg/mL of the DNA-binding fluorescent dye H33342 (10). The plates are kept at 37° C. for 30 minutes and are then washed twice with each 100 μL/well phosphaste-buffered sodium chloride solution (PBS, “phosphate buffered saline”). Subsequently, 100 μLwell PBS is added and the fluorescence is quantified at an excitation wave length of 460 nm and an emission wave length of 360 nm. The intensity of the fluorescence signal is a measure for the number of viable cells. The fluorescence background signal (dye incubation without cells) is subtracted from the raw data of the fluorescence readings and the calculation of the %-share of viable cells is made related to the fluorescence reading of cells, which were not treated with 5-FU. The fluorescence intensity of these samples is set as 100%. The dosage of 5-FU that kills 50% of the cells (ED₅₀), is determined using the same method. These experiments resulted in an ED₅₀ of 2.5 μM 5-FU for the 3T3-fibroblast cell line and an ED₅₀ of 10 mM 5-FU for human primary gingival fibroblasts. The ED₅₀-values were calculated using the nonlinear regression. This method to determine the ED₅₀ has been already described (10).

### Example 5

### Isolation of Primary Human Gingival Fibroblasts

Primary human gingival fibroblasts were obtained from gingival tissue that arises during the extraction of wisdom teeth of sound persons. This gingival tissue was cut into 1 mm² sized pieces and was then stored over night in DMEM with 10% fetal calve serum and antibiotics at 4° C. Thereafter, the tissue samples were transferred into fresh cell culture dishes, slightly dried for a better adhesion to the cell culture plates, and cultured in DMEM with 10% fetal calve serum at 37° C. and 5% CO₂ Gingival fibroiblasts are grown out of the tissue pieces after 2 to 4 days and can then be further cultured and used for the following experiments.

### Literature

1. Carreras C W, Santi D V. 1995. The catalytic mechanism and structure of thymidylate synthase. Annu Rev Biochem. 64: 721-762

2. Tong Y, Liu-Chen X, Ercikan-Abali E A, Capiaux G M, Zhao, Banerjee D, Bertino J R 1998. Isolation and characterization of thymitaq (AG337) and 5-fluoro-2deoxyuridylate-resistant mutants of human thymidylate synthase from ethyl methanesulfonate-exposed human cells. J Biol Chem. 273: 11611-11618

3. Bertino J R, Tong Y, Liu-Chen X, Banerjee D. 1997. Mutants of thymidylate synthase and uses thereof. In WO 98/33518. Sloan-Kettering Institute for Cancer Research, New York, N.Y., USA 78 pp

4. 2001. Chariot: the vehicle of the future fro protein transfection. Active Motif, Carlsbad, Calif., USA, 15 pp

5. Schwarze S R, Dowdy S F. 2000. In vivo protein transduction: intracellular delivery of biologically active proteins, compounds and DNA. Trends Pharmacol Sci. 21: 45-48

6. Dowdy S F. 2000. Protein transduction: delivery of TAT-fusion proteins into mammalian cells. Laboratory manual SFD-Lab.

7. Barsoum J G, Fawell S E, Pepinsky R B. 1992. TAT-derived transport polypeptides. In WO 94/04686. Biogen Inc, Cambridge Mass., USA, 155 pp.

8. Nagahara H, Vocero-Akbani A M, Snyder E L, Ho A, Tatham D G, Lissy N A, Becker-Hapak M, Ezhevsky S A, Dowdy S F. 1998. Transduction of full-length TAT fusion proteins into mammalian cells: TAT-p27Kip1 induces cell migration. Nat Med. 4: 1449-1452

9. Ho A, Schwarze S R, Mermelstein S J, Waksman G, Dowdy S F. 2001. Synthetic protein transduction domains: enhanced transduction potential in vitro and in vivo. Cancer Res. 61: 474-477

10. Engelmann J, Leyhausen G, Leibfritz D, Geurtsen W. 2001. Metabolic effects of dental resin components ion vitro detected by NMR spectroscopy. J Dent Res. 80: 869-875.

1. Drug for the local treatment or prevention of a mucositis or other side effects, which are induced by a chemotherapy with thymidylate-synthase-inhibtors, characterized in that:
a) the drug is designed for the local application in the gastro-intestinal tract including the oral cavity;
b) it contains at least one thymidylate-synthase-mutant either in form of a protein or a nucleic acid;
c) the thymidylate-synthase-mutant is coupled to a transducer that cause the intake into the cells to be treated;
d) the thymidylate-synthase-mutant display an intact enzyme activity, and
e) the thymidylate-synthase-mutant is resistant to thymidylate-synthase-inhibitors.

2. Drug according to claim 1, characterized in that the trancducer is a complexing agent

3. Drug according to claim 1, characterized in that the transducer is a transporting protein

4. Drug according to claim 3, characterized in that the transporting protein is the TAT-protein of HIV, the drosophila homeotic transcription factor or the herpes simplex virus type I VP22 transcription factor.

5. Drug according to claim 3 or 4, characterized in that it contains those transporting proteins, which have been optimized for their transduction efficiency by random or site-directed mutagenesis.

6. Drug according to one of the claims 1 through 5, characterized in that the thymidylate-synthase-mutant is resistant to 5-fluorouracil, fluoro-deoxyuridylate or folate analoga.

7. Drug according to one of the claims 1 through 6, characterized in that it is prepared as rinsing solution for oral application.

8. Drug according to one of the claims 1 through 6, characterized in that it is prepared as a rectally applicable administration, for instance as clysmas (enemas) or suppositories und therefore is useful for the treatment of the intestine.

9. Drug according to one of the claims 1 through 6, characterized in that it is prepared as a acid resistant oral form of administration, for instance as coated tablet or capsule, and therefore is useful for the treatment of the intestine.

10. Drug according to one of the claims 1 through 9, characterized in that it contains microorganisms, which express an appropriate thymidylate-synthase-fusion protein and therefore release the drug permanently into the intestine.

11. Drug according to one of the claims 1 through 6, characterized in that is prepared as a dried powder and will be dissolved with an appropriate diluent immediately prior to application and administration to the patient.

12. Drug according to one of the claims 1 through 10, characterized in that it contains the usual adjuvants and additives, preferentially a preservative.
Summary
Drug for the local treatment or prevention of a mucositis in the oral cavity and the gastrointestinal tract caused by chemotherapy with thymidylate-synthase-inhibitors, characterized in that a thymidylate-synthase-mutant with intact enzyme activity is applied that is resistant to thymidylate-synthase inhibitors is coupled to a transducer for the transduction into a cell.
